(19) United States
(12) Patent Application Publication
(10) Pub. No.: US 2005/0043703 A1
(43) Pub. Date: Feb. 24, 2005

(54) Slit valves for catheter tips and methods

(76) Inventor: Greg Nordgren, North Logan, UT (US)

Correspondence Address: Mr. Lynn G. Foster, 602 E. 300 S., Salt Lake City, UT, 84102, US

(21) Appl. No.: 10/647,039

(22) Filed: Aug. 21, 2003

(51) Int. Cl. A61M 31/00

(52) U.S. Cl. 604/500; 604/246

(57) ABSTRACT

Slit valves placed in normally closed distal tips at the leading end of catheter tubes are disclosed whereby tube passageway occlusion problems and body cavity interference problems are greatly reduced, if not eliminated.

### FIELD OF INVENTION

The present invention relates generally to slit valves for catheters and, more particularly to a variety of configurations of slit valves disposed in otherwise closed tips at the distal ends of catheter tubes, and related methods.

### BACKGROUND

In the past, slit valves have traditionally been used in the side walls of otherwise closed catheter tubes to infuse or aspirate fluid. Use of such side wall slit valves has been direct to infusion and aspiration of liquids in the cardiovascular systems of medical patients, infusion and aspiration of fluids in the respiratory systems of medical patients, and infusion and aspiration in other body cavities.

Disadvantageously, catheter tube side wall slit valves, when a slit of a given slit valve is open, sometimes the central passageway within the catheter tube is partially or totally occluded. Also, interference can occur between the lips (adjacent to a slit) as they are flexed outwardly and the wall of the body cavity in which the catheter tube and slit valve are disposed, which either prevents the slit valve from opening or undesirably limits the extent to which it is permitted to open.

### BRIEF SUMMARY AND OBJECTS OF THE INVENTION

In brief summary, the present invention overcomes or substantially alleviates past problems in the catheter-related slit valve field. One or more valves in a variety of forms are disposed in otherwise closed tips at the distal end of catheter tubes whereby tube passageway occlusion problems and body cavity interference problems are greatly reduced, if not eliminated.

With the foregoing in mind, it is a primary object to overcome or substantially alleviate past problems in the catheter-related slit valve field.

Another paramount object is the provision of one or more slit valves in a variety of forms, in otherwise closed tips at the distal end of catheter tubes.

A further dominate object is the provision of slit valves in the distal tips of catheter tubes by which occlusion and interference problems are greatly reduced, if not eliminated.

A further valuable object of the present invention is the provision of one or more slit valves in the distal tip of a normally closed catheter tube having one or more of the following features: Two valves on distal tip; Two aspiration valves on distal tip; Two aspiration valves and one infusion valve on the distal tip; Two valves on distal tip with an infusion valve on the apex of tip; One slit on the tip functioning as two aspiration valves and one infusion valve; Three slit valves on distal tip; Four or more slit valves on distal tip; Tip configured as a bullet, cone, pyramid, rounded, angled, dome; Tip comprising two or more flats or planar regions in which the slit valves are disposed; Slit valves in the tip having slits the same or different lengths; The interior of exterior planar regions can be flat, convex, concave, undulating, etc.; A cross sectional area at the distal tip which is the same or larger than cross sectional area at the internal diameter of tube; Stiffening ribs or strengthening elements inside tip used to prevent the tip from collapsing; Insertable over a guide wire; Comprising a material comprised of silicone rubber or polyurethane or other suitable natural or synthetic material; Applying a tip of suitable material with a catheter tube of a different material; Applies to catheters used in the vascular system, respiratory system, and other cavities within the human body; Useable with multi lumen catheter tubes with at least one slit valve at the tip aligned with each lumen, where the distal ends are or are not staggered; Reduces pressure for infusion and aspiration; Increases flow into and out of each lumen of the catheter tube; Slit valves disposed on the angled or convex portion of the rounded or tapered distal end; Distal tip formed of radiopaque material; Distal tip formed by a zero pressure molding process so that valve functions are more consistent; Distal tip formed by insert molding or connected by other processes to join the tip to a catheter tube of the same or of different material; Slit valves placed in alignment with each other; Multiple slit valves placed in parallel with each other; Multiple slits which function in unison; Multiple slits placed in alignment with one another; Multiple slit valves placed perpendicular to each other; An aspiration slit valve placed perpendicular to an infusion valve; Plural aspiration slit valves placed parallel to infusion valve; Plural aspiration slit valves and infusion slit valve utilizing a single slit; Aspiration slit valves and infusion slit valve leaving two parallel slits; All slit valves placed parallel with each other and within a plane containing the axis of the catheter tube; Opposing or offset slit valves, i.e. on different sides of the lumen, to protect against vessel wall occlusion; One slit intersecting and extending between two spaced flattened areas functioning as two, two-way valves and one, one-way valve; Two aspiration slit valves opposed or aligned on different sides of the tip to prevent vessel wall occlusion; A pyramid-shaped distal tip with one or more slit valves at one or more flat regions; and A staggered tip configuration, where the distal ends of a multi lumen catheter tube have spaced distal ending points.

These and other objects and features of the present invention will be apparent from the detailed description taken with reference to accompanying drawings.

### BRIEF DESCRIPTION OF THE DRAWINGS

FIGS. 1 through 8 are fragmentary perspectives of differently configurated, normally closed distal tips disposed at the distal end of catheter tubes, each tip having at least one tip slit valve disposed therein;

FIG. 9 is an enlarged fragmentary cross section taken along lines 9-9 of FIG. 8;

FIG. 10 illustrates an enlarged fragmentary cross section of the slit valve of the FIG. 9 in an open infusing position;

FIG. 11 is a fragmentary enlarged cross section of the slit valve of FIG. 9 showing the valve in an open aspirating condition;

FIG. 12 is a fragmentary perspective illustrating a normally closed tip on the end of a catheter tube, the tip being concentrically cone- or bullet-shaped and comprising a flat region in which one aspirating/infusing slit valve is disposed;

FIG. 13 is a fragmentary enlarged cross section taken along lines 13-13 of FIG. 12;

FIG. 14 is an enlarged perspective of a normally closed duckbill-shaped tip carried at the distal end of a catheter tube comprising a single flat region with one slit valve disposed therein;

FIG. 15 is an enlarged fragmentary perspective illustrating an eccentric bullet shaped normally closed tip disposed at the distal end of a catheter tube, the tip comprising a flat region in which a slit valve is partially disposed, the slit extending through the apex of the eccentric tip;

FIG. 16 is an enlarged fragmentary perspective of a normally closed wedge-shaped tip comprising a flat region in which two slit valves are disposed;

FIG. 17 is an enlarged fragmentary cross section taken along lines 17-17 of FIG. 16, showing the two-way slit valve lips or flaps in dotted lines open in both directions for infusion and aspiration;

FIG. 18 depicts, in fragmentary perspective, a normally closed concentric cone- or bullet-shaped tip disposed at the distal end of the catheter tube, the tip having two slit valves disposed in the rounded wall of the tip;

FIG. 19 is a fragmentary perspective of a normally closed rounded tip at the distal end of a catheter tube, the tip comprising two flat regions each with two parallel slit valves disposed therein;

FIG. 20 illustrates an eccentric bullet-shaped normally closed tip disposed at the distal end of a dual lumen catheter tube, the tip comprising two flat regions with an end of each of two two-way slit valves disposed in each flat region and bridging between the two flat regions across the apex of the tip;

FIG. 21 is an enlarged fragmentary perspective of a rounded normally closed tip disposed at the distal end of a catheter tube, the tip comprising two flat regions, each having a two-way slit valve disposed therein;

FIG. 22 is an enlarged fragmentary cross section taken along lines 22-23 of FIG. 21;

FIG. 23 is an enlarged fragmentary perspective of a normally closed rounded tip disposed at the distal end of a catheter tube, the tip comprising four flat regions, each having a radially-directed slit valve disposed therein;

FIG. 24 is an enlarged fragmentary perspective of a rounded or hemispheric tip disposed at the distal end of a catheter tube, the tip comprising six radially-directed slit valves;

FIG. 25 illustrates a hemispheric normally closed tip disposed at the distal end of a catheter tube, the tip comprising three equally spaced radially-directed slit valves;

FIG. 26 is a fragmentary cross section showing two sleeves connecting a normally closed distal tip to a distal end of a catheter tube;

FIG. 27 is a fragmentary cross section illustrating a single sleeve utilized to connect a distal normally closed tip to the distal end of a catheter tube;

FIG. 28 illustrates the diagonally disposed or beveled flat tip carried at the distal end of a catheter tube, the tip comprising a single slit valve;

FIG. 29 comprises a fragmentary perspective of a diagonally disposed or beveled tip carried at the distal end of a catheter tube, the tip comprising two parallel slit valves;

FIG. 30 illustrates a normally closed rounded tip disposed at the distal end of a multi-lumen catheter tube, the tip comprising flat regions, each aligned with one lumen with a slit valve disposed in each flat region and two additional slit valves between the two flat regions in general perpendicular relationship to the slit valves in the flat regions;

FIG. 31 is a fragmentary cross section taken along lines 31-31 of FIG. 30;

FIG. 32 is a fragmentary perspective of a multi-lumen catheter tube comprising staggered distal ends, each equipped with a slit valve;

FIG. 33 illustrates in fragmentary perspective a pyramid-shaped normally closed tip disposed at the distal end of a catheter tube;

FIG. 34 is a cross section taken along lines 34-34 of FIG. 33;

FIG. 35 is a fragmentary cross section illustrating a multi-lumen catheter tube tip with a single two-way tip slit valve in selective communication with each lumen, each valve functioning to both aspirate and infuse;

FIG. 36 is a cross section of the distal tip of a multi-lumen catheter distal tip showing four slit valves, each capable of both infusion and aspiration;

FIG. 37 illustrates a single catheter tip slit valve which opens in two different ways to provide greater infusion flow and lesser bifurcated aspiration flow through the valve;

FIG. 38 illustrates two slit valves disposed in the distal tip of the catheter tube with at least one internal reinforcing rib adjacent to each slit valve;

FIG. 39 is a fragmentary cross section showing two tip slit valves each comprising a curved interior surface which varies the thickness of the slit valves; and

FIG. 40 is a fragmentary cross section of a distal catheter tip illustrating two slit valves, each of which comprises a convex interior surface providing a varying thickness to each slit valve.

### DETAILED DESCRIPTION OF THE ILLUSTRATED EMBODIMENTS

The present invention solves or reduces past problems in the catheter-related slit valve field, applicable to the human respiratory system, the human circulatory system, and other body cavities. Apart from whether a catheter tube comprises a sidewall slit valve or does not, one or more slit valves, which may be in a variety of forms, are disposed in an otherwise closed distal end of each catheter tube whereby passageway occlusion problems and body cavity interference problems are greatly reduced, if not eliminated. The slit valves in the distal tips of catheter tubes comprise one or more slit valves, which may be either one-way or two-way valves. By two-way, it is meant that a given slit-valve both aspirates and infuses fluid into and from the associated catheter tube. By one-way, it is meant a slit valve located in the distal tip of a catheter tube which either aspirates or infuses, but does not do both.

Accordingly, the tip at the distal end of a catheter tube may comprise from one slit valve up to several slit valves to both aspirate and infuse. One or more slit valves may be located at the tip in a rounded portion thereof or in a flat region, as deemed appropriate by those having skill in the art. The distal tip may be configured as desired. For example, a bullet, a cone, a pyramid, hemispheric, angular, parabolic tip may be used. It is not necessary that all slit valves comprise a slit having the same length. Some may intersect the apex of the distal tip, where the distal tip is selected to have an apex. The slits in any of the slit valves, may have a uniform thickness or a thickness which varies, as deemed most appropriate for an intended purpose.

The preferred materials for forming the catheter tubes and the distal tips comprise silicone rubber, polyurethane and other suitable natural and synthetic elastomeric materials. The material at each slit valve must have sufficient flexibility for the lips forming the slit to flex inwardly or outwardly or both when predetermined pressure differentials are imposed thereon, in order to accommodate fluid flow in the direction desired. Treating the lips with a softening composition may take place to provide the desired flexibility.

Where multi-lumen catheter tubes are involved, typically the distal tip will comprise at least one slit valve to accommodate fluid flow into, out of, or both, for an associated lumen. The distal tip for multi-lumen catheter tubes may be longitudinally stepped or staggered.

Where it is desired to have a slit longer than the diameter of the catheter tube, the distal tip may be blunt and diagonally disposed with the slit running the full dimension of the tip or nearly so. The slits may be parallel, perpendicular, staggered, radially disposed or otherwise oriented, as deemed most appropriate by those having skill in the art. Opposing or offset slit valves located on different sides of a lumen may be used to protect against catheter tube occlusion.

The present invention provides catheter assemblies which may be inserted into a medical patient over a guide wire. Pressures required for infusion and aspiration are lower than in the past. The distal tips may be formed in any suitable fashion, zero pressure molding being presently preferred, with the distal tips being joined to the catheter tube by insert molding.

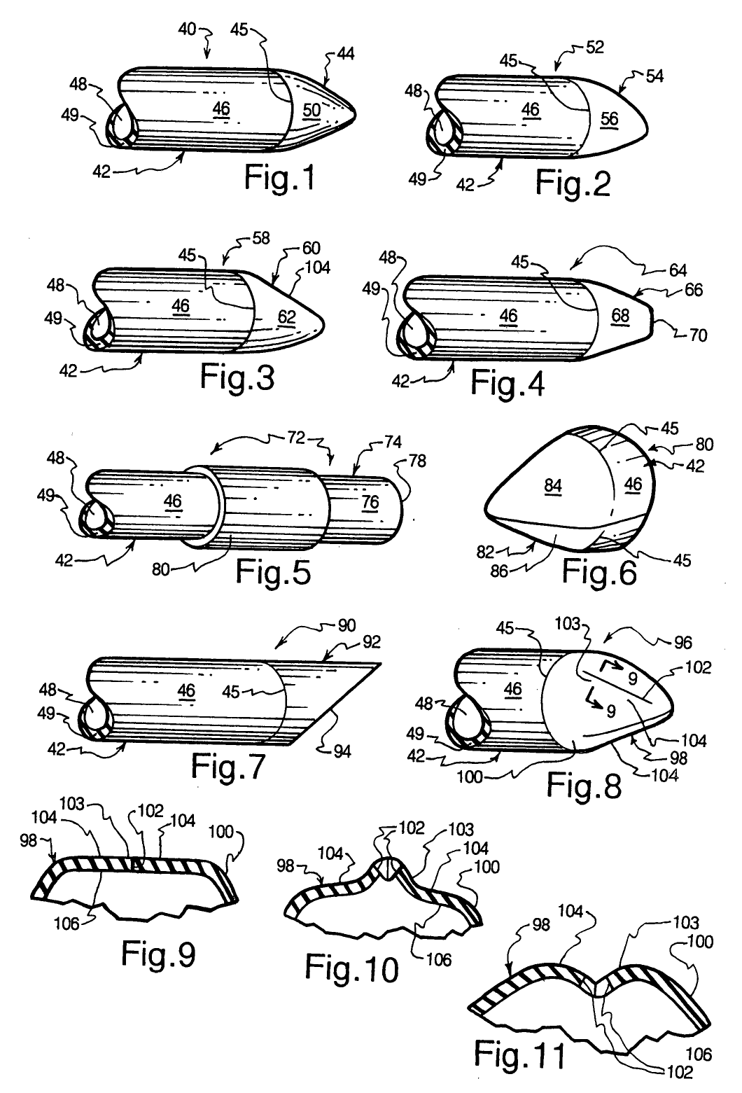

Reference is now made to the drawings wherein like numerals are used to designate like parts throughout. Any normally closed tip at the distal end of a catheter tube used for infusing, aspirating or both may be of any desired configuration. See FIGS. 1 through 8 and 33 and 34. Each of the embodiments shown in FIGS. 1 through 8 and 33 and 34 comprise the distal end of a catheter tube assembly, each comprising a catheter tube, generally designated 42, having a hollow interior of 48 forming a passageway for selective displacement of a fluid. The passageway 48 is defined within an annular wall 49 which comprises an exterior cylindrical surface 46. The distal end of each catheter tube 42 of FIGS. 1-8 and 33 and 34 is joined at interface 45 to a normally closed distal tip in which at least one slit valve is disposed. The distal tip may be above any desired configuration. The catheter tube and distal tip may be formed as one piece or separately and thereafter integrated.

FIG. 1 illustrates a concentric cone-shaped tip, generally designated 44, having an exterior surface 50.

FIG. 2 illustrates an eccentric or offset cone-shaped tip, generally designated 54, comprising an exterior surface 56.

FIG. 3 illustrates a distal tip, generally designated 60, which is in the form of a cone with an upper flat tapered face 104. The tip 60 comprises an exterior surface 62.

FIG. 4 illustrates a concentric truncated cone, generally designated 66, comprised of an exterior surface 68 and a blunt end surface 70.

FIG. 5 illustrates a sleeve-retained cylindrical tip, generally designated 74, comprised of an exterior surface 76 and a blunt, normally closed end 78, the diameter of which is the same or essentially the same as the catheter tube 42.

FIG. 6 illustrates a distal tip, generally designated 82, in the form of a duck-bill configuration comprising upper and lower exterior surfaces 84 and 86.

FIG. 7 illustrates a distal tip, generally designated 92, comprising a diagonal distal end wall 92, disposed in a single plane.

FIG. 8 illustrates a bullet-shaped tip, generally designated 98, comprising a slit-valve 104 having a slit 102 carried in a rounded portion thereof, both top and bottom.

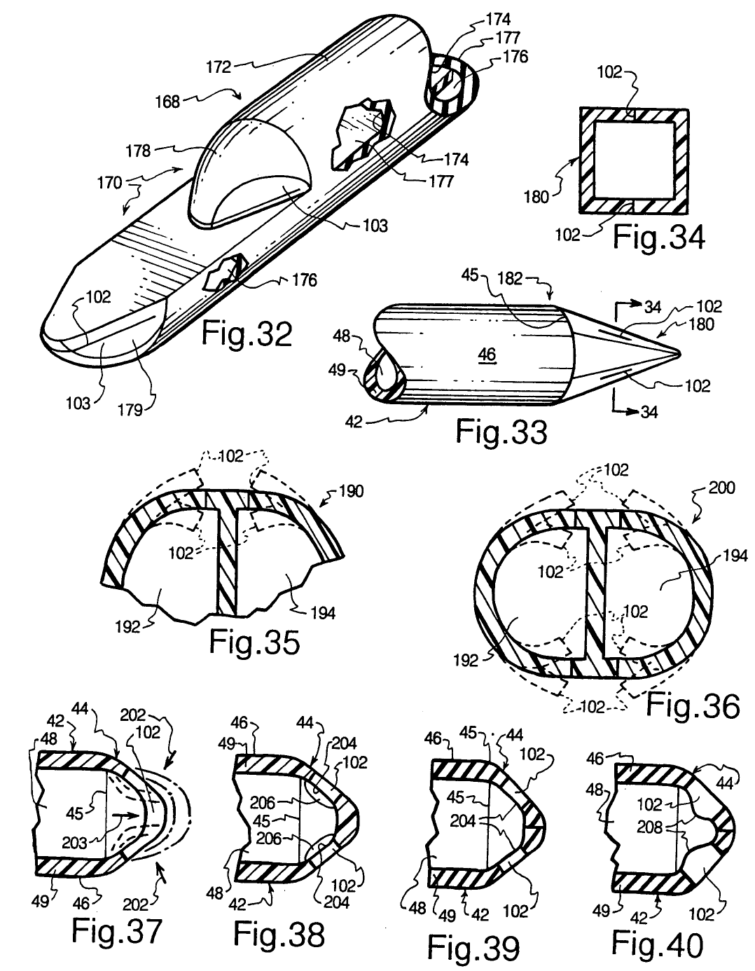

FIG. 33 illustrates a multi-lumen catheter tube comprising a distal end, generally designated 170, having a longitudinally stepped configuration so that the upper portion distally terminates before the lower portion when viewed as depicted in FIG. 33.

FIG. 34 illustrates a pyramid-shaped distal tip, generally designated 80. The catheter assembly depicted in FIGS. 1-8 and 33 and 34 are respectively designated generally by the numerals 40, 52, 58, 64, 72, 80, 90, 96, 168 and 182, respectively, and each comprise a catheter tube, generally designated 42.

While each of the distal tips 44, 54, 60, 66, 74, 82 and 98 have at least one slit valve, for simplicity no slit valve is expressly illustrated in FIGS. 1-7. The FIG. 8 tip 98 comprises parallel, opposed and co-planer slits 102 of oppositely disposed, convergently directed slit valves 102, one of which is illustrated in cross section in FIG. 9, in its normally closed condition. The slit 102 illustrated in FIG. 9 comprises part of slit valve 103 having flexible lips disposed in a portion of a flat region 104 of the tip 98. The tip 98 is illustrated as comprising a wall 100 illustrated as being of uniform thickness defined by exterior flat surface 104 and interior surface 106. As illustrated in FIG. 9, the two-way slit valve 103 is in its normally closed position with the shoulders of the opposed lips defining the slit 102 being imperviously contiguous and aligned one with the other. The closed condition illustrated in FIG. 9 is the normal position when the distal tip 98, with the associated distal end of the catheter tube 42 being indwelling within a medical patient. The slit 102 remains closed unless and until a pressure differential exists of selected magnitude between the interior pressure and exterior pressure of the tip such that the lips of the valve 103 are flexed outwardly to open the slit 102 to accommodate infusion, as illustrated in FIG. 10 or inwardly, as illustrated in FIG. 11, to accommodate aspiration.

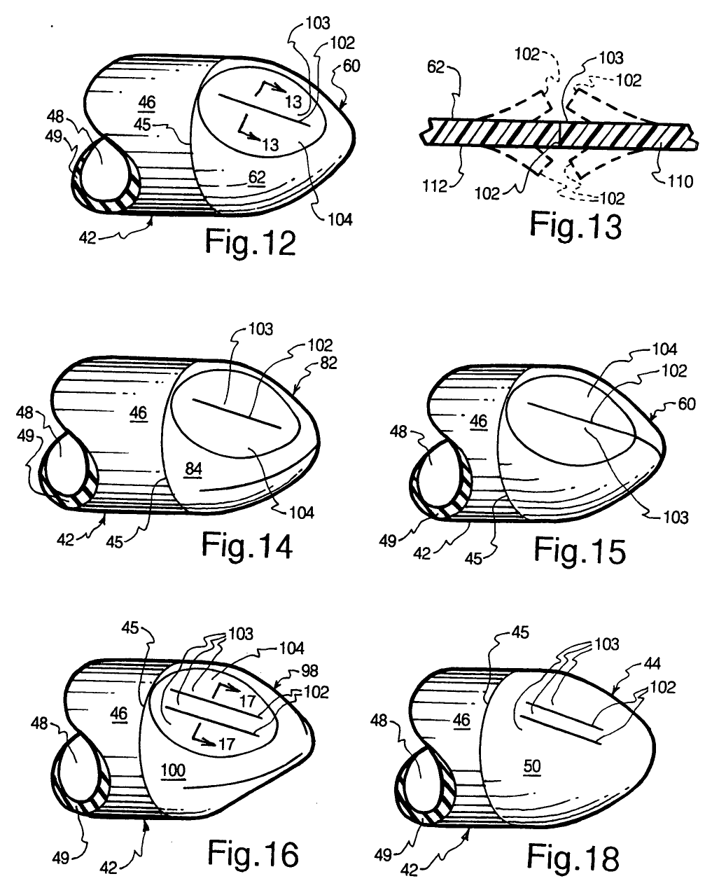

With reference to FIG. 12, the distal tip 60 is shown to comprise one or more exterior flat surfaces 104 (aka flat 104) which is centrally interrupted by slit 102 comprising part of slit valve 103.

FIG. 13 illustrates that slit 102 of FIG. 12 is a three position, two-way valve. The first normally-closed position is illustrated in solid lines showing the slit impervious to fluid flow therethrough. The upper dotted representation depicts the slit 102 being in an outwardly open, infusing position, while the lower dotted lines illustrate the slit in its inwardly open, aspirating position.

FIG. 14 illustrates the distal tip 82. Tip 82 is illustrated as comprising one or more flat regions 104 with a slit 102 centrally disposed and contained therein. It is in a plane which contains the longitudinal axis of the catheter tube and is aligned with the apex of the tip. Slit 102 forms a part slit valve 103. This figure also shows the slit 102 extending across the apex of the tip for infusion and passage of a guide wire for placement purposes.

FIG. 15 illustrates the distal tip 60 configured to comprise one or more flat regions 104 and a slit centrally disposed in the flat 104 and extending to and around the apex of the tip 60. Each slit functions as two aspiration valves and one infusion valve.

It should be clear from the foregoing that the slit valves 103 illustrated in FIGS. 12, 14 and 15 are two-way, aspirating and infusing valves, each of which opens when pre-determined thresholds of infusing and aspirating pressure differentials take place between the pressure inside the catheter tube 42 and the pressure outside the catheter tube 42 when in-dwelling within a body cavity of a medical patient.

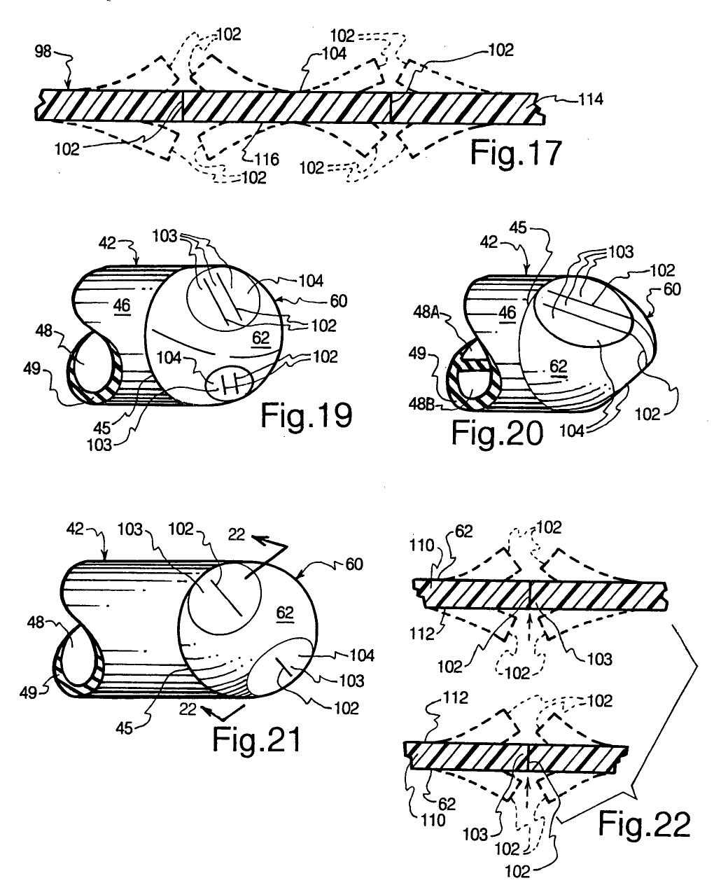

FIG. 16 illustrates distal tip 98 configured so as to comprise a flat 104 in which two parallel longitudinally-directed slits 102 comprising adjacent slit valves 103 are disposed, each being generally directed toward the apex of the distal tip 98. The aspirating and infusing functions of the two valves 102 in FIG. 16 is illustrated in lower and upper dotted lines respectively in FIG. 17.

FIG. 18 illustrates distal tip 44 configured to comprise two slits 102 each forming part of a slit valve 103, with the slits oriented and parallel similar to FIG. 17, except slits 104 in FIG. 18 are disposed in the rounded surface of the tip 44 and not in a flat.

FIG. 19 illustrates the distal tip 60 configured to comprise two flats 104, oppositely disposed, each with two parallel slits 102 therein whereby any of the slits may be used to aspirate and any to infuse.

FIG. 20 illustrates a multi-lumen catheter tube 42 with lumens 48A and 48B and a tip 60 comprised of two flats 104, disposed respectively in converging planes each lumen containing one slit 102 comprising two slit valves wherein the slits run through one flat 104 around the apex of the tip and through the second flat. Each single slit operates as two aspiration valves and one infusion valve.

FIG. 21 illustrates rounded distal tip 60 comprised of two spaced flats 104, each with a slit 102, comprising part of a slit valve 103, disposed therein, both valves are for aspirating and infusion.

FIG. 22 illustrates that the top slit 102 of the tip of FIG. 21 accommodates infusion, the infusing outwardly open position of slit 102 illustrated as being flexed outwardly as shown by the dotted lines at the top of FIG. 22. The lower slit 102 accommodates aspiration when inwardly open into the lower dotted line depiction. However, either valve will accommodate both infusion or aspiration.

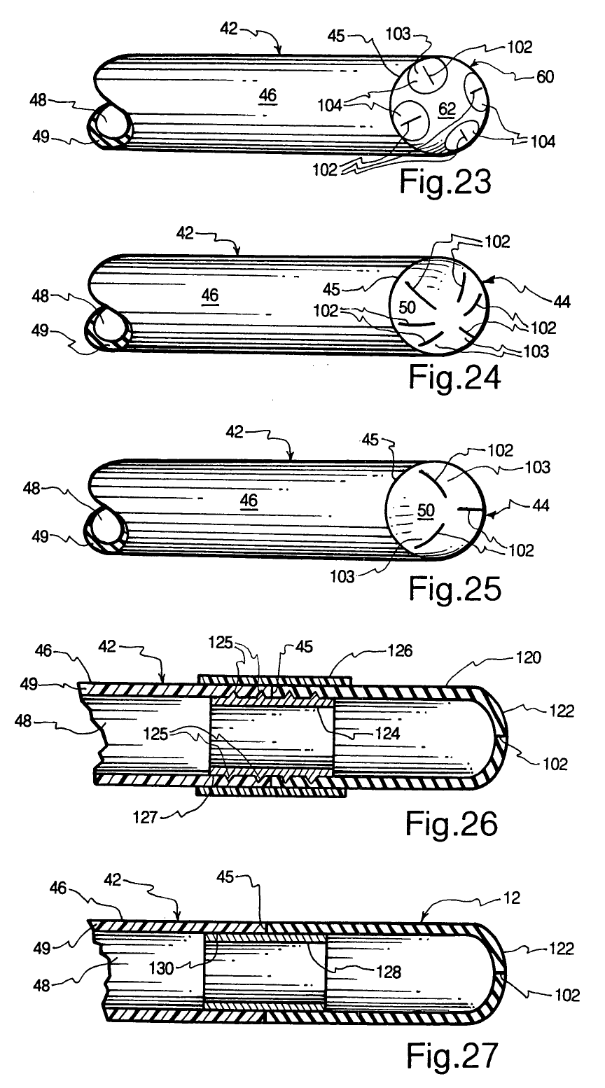

FIGS. 23, 24 and 25 illustrate, respectively, distal tips 60, 44 and 44, shown to be rounded with four, six and three radially extending slits 102, respectively, accommodating infusion and aspiration in each embodiment. The embodiment of FIG. 23 shows the radially disposed slits 102 being contained in spaced flats 104, while FIGS. 24 and 25 show the slits in the rounded wall of the tip.

Reference is now made to FIGS. 26 and 27 which illustrate utilization of an elongated valved tip 120 at the distal end of catheter tube 42, secured in place, respectively, by a pair of sleeves 124 and 126 (FIG. 26) and a single sleeve (FIG. 27). In FIG. 26, the interior sleeve 124 comprises a smooth cylindrical interior surface, while the exterior surface comprises a plurality of annular serrations or teeth 125, which protrude into the material comprising the distal end of the catheter tube and the material comprising the proximal end of the valved tip 120. Preferably the exterior sleeve 126 is either press fit into the position illustrated in FIG. 26 or shrinkage techniques are used to reduce the diameter of the sleeve 126 once positioned around interfaced 45 to permanently and imperviously connect the catheter tube 42 to the tip 120. In the configuration of FIG. 27, a suitable adhesive, bonding compound, or other connecting technique, creates an impervious union at interface 130, interposed between the exterior surface of the sleeve 128 and the adjacent interior surfaces of the catheter tube 42 and the valved tip 120.

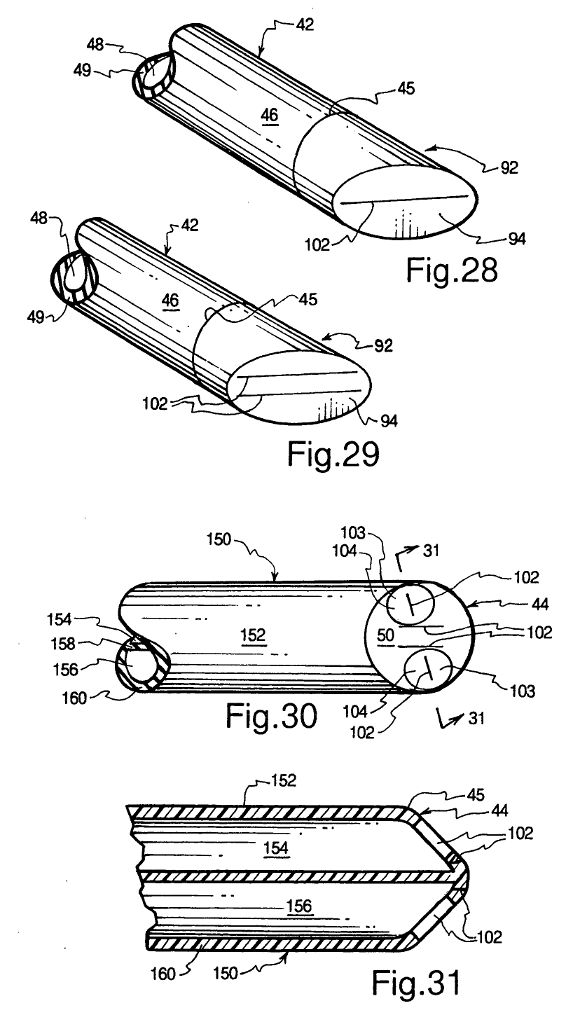

Reference is now made to FIGS. 28 and 29 which respectively illustrate tip 92 with diagonally disposed planar distal wall 94. FIG. 28 illustrates a single two-way slit 102, while FIG. 29 illustrates two slits along the diagonal end wall 94, both for aspirating and infusing. Using this configuration, the length of the diagonal slit or slits 102 may be greater dimensionally than the diameter of the associated catheter tube 42.

Reference is now made to FIGS. 30 and 31, which illustrate a multi-lumen catheter tube 150. While catheter tube 150 is illustrated as comprising two lumens 154 and 156, more than two could comprise the catheter tube.

The lumens 154 and 156 are imperviously separated one from the other by an interior divider wall 158, the cylindrical wall 160 of the catheter tube 150 defining the exterior of both lumens. The catheter tube 150 terminates at its distal end in normally closed tip 44, illustrated as comprising two flats 104, each equipped with a radially disposed slit 102 and two transversely disposed slits 102 located between the two flats 104. The arrangement of the four slits is best seen in FIG. 31 with one of the two slits aligned with lumen 154 functioning to aspirate and the other to infuse, while the two slits 102 aligned with lumen 156 respectively function to infuse and aspirate.

FIG. 32 illustrates that a distal tip in accordance with the present invention may comprise staggered or stepped elements or half ends. FIG. 32 illustrates a multi-lumen catheter assembly 168 comprising a catheter tube 172. Catheter tube 172 is illustrated as comprising two lumens 174 and 176 separated by an impervious interior wall 177. The assembly 178 comprises a stepped distal tip 170, which comprises two half tips 178 and 179, each comprising a slit 102 forming part of a slit valve 103. The slits 102 are at longitudinally spaced locations, each in communication with the lumen in alignment therewith.

FIG. 33 illustrates a pyramid-shaped tip, generally designated 180, with a slit 102 disposed in at least one of the planar portions of the tip. However, more than one and, if desired, all of the flat surfaces of the tip 180 may comprise one or more slits 102 to accommodate infusion and aspiration, in the manner explained above. Two such slits are shown in FIG. 34.

FIG. 35 illustrates a further catheter tube distal tip configuration, generally designated 190, the catheter tube and the distal tip comprising two lumens, each equipped with a two-way aspirating and infusing slit valve 102.

FIG. 36 is similar to the embodiment of FIG. 35 except the tip configuration, generally designated 200, comprises two two-way slit valves 102 for each lumen 192 and 194, each slit valve accommodating both aspiration and infusion into and out of the associated lumen.

Reference is now made to FIG. 37 wherein the single around-the-apex slit 102 is illustrated as accommodating creation of two aspirating flow paths when the lips forming the slit valve are displaced into the dotted line position, aspiration occurring in the direction of arrows 202. The slit 102 also creates a single larger opening, illustrated in the phantom lines in FIG. 37, to accommodate infusion of a larger quantity of fluid per unit of time.

Reference is made now to FIG. 38 which illustrates that the slits 102 may have a varying thickness along the length thereof caused by curvilinear face 204 and may be reinforced interiorly by a suitable structure such as ribs 206.

FIG. 39 illustrates that the thickness of the slits 102 may vary across the length of any given slit 102, without the need for internal tip reinforcement.

FIG. 40 illustrates that the variation in thickness along the length of the slit valve 102 may increase from end to end rather than decrease. Specifically, the increase in wall thickness is illustrated at 208.

The invention may be embodied in other specific forms without departing from the spirit of the central characteristics thereof. The present embodiments therefore are to be considered in all respects as illustrative and not restrictive, the scope of the invention being indicated by the appended claims rather than by the foregoing description, and all changes which come within the meaning and range of equivalency of the claims are therefore intended to be embraced therein.

1. A slit valve catheter comprising:
a catheter tube comprising at least one lumen;
a closed distal tip at the distal end of the catheter tube;
at least one normally closed slit valve comprising a slit which normally prohibits fluid flow but which opens when indwelling under a predetermined fluid pressure differential to accommodate fluid flow through the open slit.

2. A slit valve catheter according to claim 1 wherein the catheter tube comprises a plurality of internal lumens with a tip slit valve aligned with each lumen.

3. A slit valve catheter according to claim 1 wherein the tip comprises a concentric generally cone-shaped wall.

4. A slit valve catheter according to claim 1 wherein the tip comprises an asymmetric eccentric generally cone-shaped wall.

5. A slit valve catheter according to claim 1 wherein the tip comprises a concentric generally cone-shaped wall comprising at least one flat area in which the at least one slit valve is disposed.

6. A slit valve catheter according to claim 5 wherein the at least one flat area comprises at least two flat areas, each with at least one slit valve there disposed.

7. A slit valve catheter according to claim 1 wherein the tip comprises a blunt distal wall in which at least one slit valve is disposed.

8. A slit valve catheter according to claim 7 wherein part of the tip convergently tapers between the catheter tube and the blunt distal wall.

9. A slit valve catheter according to claim 1 wherein the tip comprises a flat beveled wall in which at least one slit valve is disposed.

10. A slit valve catheter according to claim 1 wherein the tip comprises a duckbill configuration.

11. A slit valve catheter according to claim 1 wherein at least one slit valve comprises a two way valve comprising lips adjacent to the slit which when indwelling selectively flex inwardly and outwardly depending on the pressure differential to aspirate and infuse.

12. A slit valve catheter according to claim 1 wherein the catheter tube is formed of a material selected from the group consisting of urethane, silicone rubber and natural and synthetic elastomers.

13. A slit valve catheter according to claim 1 wherein the tip is formed substantially of a material selected from the group consisting of urethane, silicone rubber and natural and synthetic elastomers.

14. A slit valve catheter according to claim 1 wherein the slit valve is formed of a material selected from the group consisting of urethane, silicone rubber and natural or synthetic elastomeric material treated to enhance flexure from the normally closed to an open position.

15. A slit valve catheter according to claim 1 wherein the catheter tube and the tip are formed as one piece.

16. A slit valve catheter according to claim 1 wherein the catheter tube and tip are first formed as two pieces and then integrated.

17. A slit valve catheter according to claim 16 wherein the catheter tube and tip are integrated by at least one overlapping sleeve.

18. A combination comprising a normally closed distal catheter tip comprising a tip wall and at least one normally closed but selectively openable slit valve disposed in the tip wall.

19. A combination according to claim 18 wherein the tip configuration is either symmetric or asymmetric and selected from the group consisting of pyramid, rounded, parabolic and cone shapes.

20. A combination according to claim 18 wherein the tip configuration is either symmetric or asymmetric and selected from the group consisting of dome, transversely blunt, diagonally disposed blunt, hemispheric and balloon-shaped.

21. A combination according to claim 18 wherein the at least one slit valve is selected from the group consisting of two or more one-way valves and one or more two-way valves.

22. A combination according to claim 18 wherein the slit of the slit valve varies in thickness along its length.

23. A combination according to claim 18 wherein the slit valve is internally structurally reinforced.

24. A combination according to claim 18 wherein the catheter is multi-lumen and the tip distally terminates in longitudinally spaced staggered end elements, each having at least one slit valve aligned with one lumen.

25. A combination according to claim 18 wherein the slit of the slit valve traverses around an apex of the tip.

26. A combination according to claim 18 wherein the tip comprises a beveled blunt end with the slit valve disposed therein such that the length of the slit is greater than the diameter of the catheter.

27. A combination according to claim 18 wherein the slit valve comprises a two-way valve which flexes to create a large infusion flow path and a small aspiration flow path.

28. A method of using a normally closed catheter tube comprising a distal normally closed tip having at least one normally closed slit valve comprising the acts of:
placing the tip indwelling in a patient;
creating a fluid pressure differential across the normally closed slit valve such that the slit valve flexes to an open position to accommodate fluid flow through the slit valve between an interior and an exterior of the indwelling tip.

29. A method of making a normally closed catheter comprising the acts of:
forming a catheter tube comprising at least one lumen and a distal end;
integrally forming a tip at the distal end of the catheter tube;
causing at least one slit valve to be formed in the tip.
